(19) United States
(12) Patent Application Publication
(10) Pub. No.: US 2003/0027867 A1
(43) Pub. Date: Feb. 6, 2003

(54) Use of R-NSAID compounds for anti-HIV treatment

(75) Inventor: Kenton Zavitz, Salt Lake City, UT (US)

Correspondence Address: MYRIAD GENETICS INC., LEGAL DEPARTMENT, 320 WAKARA WAY, SALT LAKE CITY, UT, 84108, US

(73) Assignee: Myriad Genetics, Incorporated, Salt Lake City, UT (US)

(21) Appl. No.: 10/186,371

(22) Filed: Jun. 28, 2002

Related U.S. Application Data
(60) Provisional application No. 60/302,533, filed on Jun. 29, 2001.

(51) Int. Cl. A61K 31/192

(52) U.S. Cl. 514/569; 514/570

(57) ABSTRACT

The present invention provides a method for treating a patient with HIV infection. The method includes administering to a patient a composition containing a therapeutically effective amount of a R-NSAID or a pharmaceutically acceptable salt or ester thereof. The composition is substantially free of S-NSAID.

### RELATED U.S. APPLICATION

This application claims the benefit of U.S. Provisional Application Serial No. 60/302,533 filed on Jun. 29, 2001, which is incorporated herein by reference in its entirety.

### FIELD OF THE INVENTION

The present invention relates generally to treatment and prevention of viral infection, and particularly to compositions and methods useful in the treatment of HIV infection and AIDS.

### BACKGROUND OF THE INVENTION

Human immunodeficiency virus (HIV) infection causes the acquired immunodeficiency syndrome (commonly known as AIDS). HIV is a retrovirus that primarily infects T cells expressing the CD4 glycoprotein, i.e., CD4⁺ T-cells, which are also known as helper T-cells. HIV virus multiplies in helper T-cells and quickly destroys the host helper T-cells, resulting in cellular immunity depression and leaving the infected patient susceptible to opportunistic infections, malignancies and various other pathological conditions. Ultimately, HIV infection can cause depletion of helper T-cells and collapse of a patient's immune defenses. Not surprisingly, HIV-infected individuals and AIDS patients typically develop AIDS-related conditions such as AIDS-related complex (ARC), progressive generalized lymphadenopathy (PGL), dementia, tropical paraparesis, Kaposi's sarcoma, thrombocytopenia purpurea, herpes infection, cytomegalovirus infection, Epstein-Barr virus related lymphomas among others. In any case, the HIV viruses in an infected individual are infectious and can be transmitted to other people through blood transfusion or sexual contacts.

There has been a great deal of effort in the past fifteen years or so in developing pharmaceutical compounds for treating HIV infection and AIDS. The therapeutic approaches have been focused on a limited number of drug targets, namely HIV reverse transcriptase, HIV protease, and HIV integrase. A number of reverse transcriptase inhibitors and protease inhibitors have been developed or marketed. Examples of nucleoside reverse transcriptase inhibitors include Zidovudine, Stavudine, Lamivudine, and ddI. Examples of non-nucleoside reverse transcriptase inhibitors include Efavirenz, Delavirdine, and Abacavir. In addition, a number of HIV protease inhibitors are commercially available including Ritonavir, Nelfinavir, Indinavir and Saquinavir.

However, HIV typically undergoes active mutations as it multiplies. In addition, there are extensive genetic variations in HIV partly due to the high mutation rate. Therefore, mutations in HIV reverse transcriptase and protease arise frequently in infected individuals and render the virus resistant to the inhibitor administered to the individuals. Combination therapy has been developed in which a combination of different anti-HIV inhibitors is administered to a patient. However, viral resistance to combination therapies still frequently develops.

In addition, many of the anti-HIV compounds known in the art have other serious drawbacks. For example, the reverse transcriptase inhibitors such as AZT and ddI are fairly toxic and cause serious side effects in patients treated with such compounds.

Therefore, although limited success for controlling HIV infection and AIDS has been achieved with previously developed anti-HIV compounds, there is a need for alternative therapeutic approaches that overcome the shortcomings of currently available drugs.

### SUMMARY OF THE INVENTION

The present invention provides a method for treating or preventing viral infection, particularly HIV infection and AIDS which includes administering to a patient a composition containing a therapeutically effective amount of the R-enantiomer of an NSAID and substantially free of the corresponding S-enantiomer. Suitable R-NSAIDs may include a R-enantiomer of ketoprofen, flurbiprofen, naproxen, tiaprofenic acid, suprofen, etodolac, carprofen, ketorolac, pirprofen, indoprofen, benoxaprofen, and the like.

Unlike S-NSAIDs, which are known to be associated with various side effects, R-NSAIDs do not cause any significant adverse reactions. Preferably R-flurbiprofen is used as the active ingredient. Flurbiprofen belongs to the 2-arylpropionic acid class of NSAID and has been used as anti-flammatory agents and analgesics. While S-flurbiprofen causes significant gastrointestinal mucosal damage and other serious side effects, R-flurbiprofen does not cause any significant side effect even at very high concentrations. Accordingly, the method of the present invention provides a novel approach for treating HIV infection and AIDS that is not associated with significant adverse side effects.

In addition, R-NSAIDs such as R-fluribiprofen are not believed to fall within any currently known classes of anti-HIV compounds. They can be used as alternative treatment of HIV infection and AIDS. In addition, they may be included in combination therapies with other classes of anti-HIV compounds such as reverse transcriptase inhibitors and protease inhibitors. Because of their distinct HIV inhibition mechanisms and low side effects, the R-NSAIDs, particularly R-flurbiprofen, can be especially desirable in combination therapies with reverse transcriptase inhibitors, protease inhibitors, and the like.

Preferably, a composition comprising the R-enantiomer of 3-fluoro-4-phenylhydratropic acid (R-flurbiprofen) or a pharmaceutically acceptable salt or ester thereof is administered to a patient in need of treatment. The composition is substantially free of S-flurbiprofen.

In one embodiment, an ester of R-flurbiprofen is used as an active compound. Preferably, an alkyl ester of 1-8, or 1-6 carbon atoms is included in the composition as an active ingredient. For example, the esters can be the methyl, ethyl, propyl, isopropyl, butyl, isobutyl, tert-butyl, pentyl, and octyl esters.

In another embodiment, a pharmacologically acceptable salt of R-flurbiprofen is included in the composition of the present invention as an active ingredient. Examples of such a pharmacologically acceptable salt include the alkali metal salts, alkaline earth salts, and ammonium salts.

In another embodiment, the composition of the present invention includes, in addition to R-flurbiprofen or a pharmaceutically acceptable salt or ester thereof, one or more other anti-HIV compounds. Examples of such compounds include Zidovudine, Lamivudine, Stavudine, DMP-266, Ritonavir, Nelfinavir, Abacavir, Indinavir, Tenofovir, 141-W94, Delavirdine, Indinavir, Saquinavir, and HIV fusion inhibitors such as T-20, or a pharmaceutically acceptable salt or ester thereof.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a graph showing the effect of R-flurbiprofen at various concentrations on HIV viral propagation in cell culture and on cell viability in the cell culture;

FIG. 2 shows the effect of AZT at various concentrations on HIV viral propagation in cell culture and on cell viability in the cell culture.

### DETAILED DESCRIPTION OF THE INVENTION

As used herein, the term “HIV infection” generally encompasses infection of a host animal, particularly a human host, by the human immunodeficiency virus (HIV) family of retroviruses including, but not limited to, HIV I, HIV II, HIV III (a.k.a. HTLV-III, LAV-1, LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or prophylaxis of the AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD⁴⁺ T cells), and AIDS (which is defined by more serious AIDS-defining illnesses and/or a decline in the circulating CD4 cell count to below a level that is compatible with effective immune function). In addition, “treating or preventing HIV infection” will also encompass treating suspected infection by HIV after suspected past exposure to HIV by e.g., contact with HIV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HIV infection” may also encompass treating a person who has not been diagnosed as having HIV infection but is believed to be at risk of infection by HIV.

The term “treating AIDS” means treating a patient who exhibits more serious AIDS-defining illnesses and/or a decline in the circulating CD4 cell count to below a level that is compatible with effective immune function. The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Thus, the term “preventing AIDS” as used herein means preventing in a patient who has HIV infection or is suspected to have HIV infection or is at risk of HIV infection from developing AIDS (which is characterized by more serious AIDS-defining illnesses and/or a decline in the circulating CD4 cell count to below a level that is compatible with effective immune function) and/or AIDS-related conditions.

The term “substantially free of” when used in connection a composition containing a R-NSAID, particular R-flurbiprofen, means that the composition does not contain the S-enantiomer of the R-NSAID in any significant amount that is sufficient to elicit any significant adverse effect in a patient to whom the composition is administered. Typically, the ratio between R-NSAID and its S-enantiomer in the composition being administered is at least 70:30 by weight or 80:20 by weight, preferably at least 90:10 by weight, more preferably at least 95:5 by weight, and most preferably at least 99:1 by weight.

The present invention provides a method for treating or preventing HIV infection or AIDS by administering to a patient in need of such treatment a composition containing a therapeutically effective amount of R-NSAID. The composition is substantially free of the S-enantiomer of the R-NSAID.

A great number of NSAIDs are known in the art. Certain NSAIDs, such as ketoprofen and flurbiprofen are arylpropionic acids, while others are cyclized derivatives of arylpropionic acids, arylacetic acids, thiazinecarboxamides, etc. Depending on the structure of a particular NSAID, the compound may or may not exhibit chirality, i.e., may not have R- and S-enantiomers. Accordingly, the active compounds useful in the present invention are the R-enantiomers of (1) those known NSAIDs that exhibit chirality, or (2) NSAID derivatives that exhibit chirality.

In one embodiment, the R-NSAID employed in the compositions is an arylpropionic acid or a pharmaceutically acceptable salt or ester thereof, in particular a compound selected from the group consisting of R-flurbiprofen, R-ketoprofen, R-naproxen, R-tiaprofenic acid, R-suprofen, R-carprofen, R-pirprofen, R-indoprofen, and R-benoxaprofen. The R-NSAID can also be a cyclized derivative of arylpropionic acid, such as R-ketorolac, or an arylacetic acid, such as R-etodolac.

In a preferred embodiment, a pharmaceutical composition that contains R-flurbiprofen and is substantially free of S-flurbiprofen is administered to a patient to treat or prevent HIV infection or AIDS. Preferably, the ratio between R- flurbiprofen and its S-enantiomer in the composition being administered is at least 70:30 by weight or 80:20 by weight, preferably at least 90:10 by weight, more preferably at least 95:5 by weight, and most preferably at least 99:1 by weight.

The chemical formula of some of the known NSAIDs with chirality are shown below:

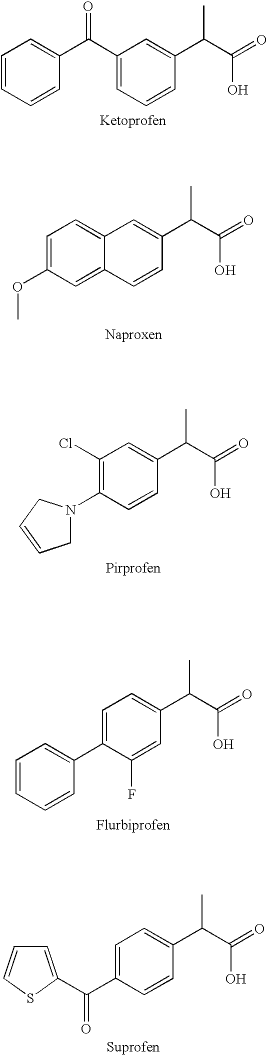

Pharmaceutically acceptable salts of the R-NSAIDs may also be employed. The term “pharmaceutically acceptable salts” refers to salts prepared from pharmaceutically acceptable, non-toxic acids or bases. Examples of such salts include, but are not limited to, alkali metal salts, alkaline earth salts, and ammonium salts. Thus, suitable salts may be salts of aluminum, calcium, lithium, magnesium, potassium, sodium and zinc. In addition, organic salts may also be used including, e.g., salts of lysine, N,N′-dibenzylethylenediamine, chloroprocaine, choline, diethanolamine, ethylenediamine, meglumine (N-methylglucamine), procaine and tris.

In addition, a R-NSAID in the pharmaceutical composition can also be selected from various esters of the R-NSAIDs, particularly esters of R-enantiomers of arylpropionic acid NSAIDs, esters of R-enantiomers of NSAIDs that are cyclized derivative of arylpropionic acid, and esters of R-enantiomers of arylacetic acid NSAIDs. Preferably, the esters are alkyl esters of from 1 to 8, more preferably from 1 to 6 carbon atoms, for example, methyl, ethyl, propyl, isopropyl, butyl, isobutyl, tert-butyl, pentyl, and octyl esters. In addition, various other derivatives such as amides and alcohols and other derivatives of the NSAIDs can also be used so long as they can be metabolized in the patient body to form the corresponding acid form of the R-NSAIDs.

Various pharmaceutical uses of the above-described NSAIDs and methods making them are known in the art. For example, flurbiprofen is described in U.S. Pat. Nos. 3,755,427; 4,230,724; 5,556,638; 5,955,504; 5,981,592; and 6,160,018, all of which are incorporated herein by reference. Ketoprofen is described in U.S. Pat. No. 3,641,127, which is incorporated herein by reference. Ketorolac, another chiral NSAID, is described in U.S. Pat. No. 4,089,969 and Muchowski et al., J. Med. Chem., 28(8):1037-1049 (1985), both of which are incorporated herein by reference.

In addition, a large number of NSAIDs are commercially available either in the form of racemic mixtures or as optically pure enantiomers. Optically pure R-NSAIDs can be obtained from the racemic mixtures according to well-known methods. See, e.g., U.S. Pat. No. 5,331,000 (R-ketoprofen) and U.S. Pat. No. 5,382,591 (R-ketorolac), the contents of each of which are incorporated herein by reference.

Racemates of ketoprofen, flurbiprofen, etodolac, suprofen, carprofen, indoprofen and benoxaprofen can be obtained through Sigma Chemical Co. R-naproxen can also be obtained as the sodium salt from Sigma Chemical Co. Additionally, many R-enantiomers including R-ketoprofen, R-flurbiprofen and R-ketorolac are available, e.g., from Sepracor, Inc. In addition, R-flurbiprofen is also commercially available from Catalytica Pharmaceutical Inc., Greenville, N.C. R-etodolac is available from Wyeth-Ayerst. R-tiaprofenic acid is available through Roussel (France, Canada, Switzerland, Spain, Denmark, Italy). R-suprofen is manufactured by McNiel Pharmaceuticals. R-carprofen is available from Roche. R-pirprofen is available through Ciba (France, Belgium, Denmark). R-indoprofen can be obtained through Carlo Elba (Italy, U.K.). R-benoxaprofen is manufactured by Eli Lilly Co.

R-NSAID derivatives can also be made by altering the structures of known R-NSAIDs. Certain derivatives of flurbiprofen is disclosed in Bayly et al., Bioorg. Med. Chem. Lett., 9:307-312 (1999), which is incorporated herein by reference. Synthesis of other NSAID derivatives is reviewed in Dewitt, Mole. Pharm., 55:625-631 (1999). For example, derivatives of R-flurbiprofen, R-fenoprofen, R-ketoprofen, or R-carprofen can be provided by altering one or more of the followings: (1) altering the position of the propionic acid group, (2) the position or type of substituents (other than the propionic acid group) on either of the phenyl rings, (3) the bond connecting the two phenyl rings, and (4) the acetic acid group (e.g., to carboxylic acid group, or to esters). Methods for modifying other R-NSAIDs such as R-naproxen, R-tiaprofenic acid, R-suprofen, R-pirprofen, R-indoprofen, R-benoxaprofen, R-ketorolac, R-etodolac and the like should be apparent to skilled artisan apprised of the above disclosure.

Thus, the present invention also provides a method for identifying an antiviral, particularly anti-HIV agent, which includes the following steps: (1) providing a R-NSAID derivative or R-NSAID, and (2) determining the effect of the R-NSAID or R-NSAID derivative on viral propagation, particularly HIV viral propagation. Various cell-based assays or human clinical testing generally known in the art can be employed in determining the anti-viral efficacy of the R-NSAIDs or R-NSAID derivatives.

NSAIDs are a novel class of anti-HIV compounds distinct from other commercially available compounds. While not wishing to be bound by any theory or hypothesis, it is believed that NSAIDs inhibit HIV through a mechanism distinct from those of the anti-HIV compounds known in the art which typically are either protease inhibitors or reverse transcriptase inhibitors. Therefore, it may be desirable to employ combination therapies to administer to a patient both a R-NSAID according to the present invention and another anti-HIV compound of a different class. However, it is to be understood that such other anti-HIV compounds should not interfere with or adversely affect the intended effects of the active compounds of this invention. In this combination therapy approach, the two different pharmaceutically active compounds can be administered separately or in the same pharmaceutical composition. Compounds suitable for use in combination therapy with the R-NSAIDs according to the present invention include, but are not limited to, HIV protease inhibitors, nucleoside HIV reverse transcriptase inhibitors, non-nucleoside HIV reverse transcriptase inhibitors, HIV integrase inhibitors, HIV fusion inhibitors, immunomodulators, and vaccines.

Examples of nucleoside HIV reverse transcriptase inhibitors include 3′-Azido-3′-deoxythymidine (Zidovudine, also known as AZT and RETROVIR®), 2′, 3′-Didehydro-3′-deoxythmidine (Stavudine, also known as 2′, 3′-dihydro-3′-deoxythymidine, d4T, and ZERIT®), (2R-cis)-4-Amino-1 -[2-(hydroxymethyl)-1,3-oxathiolan-5-yl]-2(1H)-pyrimidinone (Lamivudine, also known as 3TC, and EPIVIR®), 2′, 3′-dideoxyinosine (ddI), and 9-[(R)-2-[[bis[[isopropoxycarbonyl)oxy]methoxy]phosphinyl]methoxy]propyl] adenine fumarate (Tenofovir disoproxil fumarate, also known as Viread™).

Examples of non-nucleoside HIV reverse transcriptase inhibitors include (−)-6-Chloro-4-cyclopropylethynyl-4-trifluoromethyl-1,4-dihydro-2H-3,1-benzoxazin-2-one (efavirenz, also known as DMP-266 or SUSTIVA®) (see U.S. Pat. No. 5,519,021), 1-[3-[(1-methylethyl)aminol]-2-pyridinyl]-4-[[5-[(methylsulfony)amino]-1H-indol-2yl]carbonyl]piperazine (Delavirdine, see PCT International Patent Application No. WO 91/09849), and (1S ,4R)-cis-4-[2-amino-6-(cycloprpoylamino)-9H-purin-9-yl]-2-cyclopentene-1-methanol (Abacavir).

Examples of protease inhibitors include [5S-(5R*,8R*, 10R*,11R*)]-10-hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid 5-thiazolylmethyl ester (Ritonavir, marketed by Abbott as NORVIR®), [3S-[2(2S*,3S*),3a,4ab,8ab]]-N-(1,1-dimethylethyl)decahydro-2-[2-[2-hudroxy-3-[(3-hydroxy-2-methylbenzoyl)amino]-4-(phenylthio)butyl]-3-isoquinolinecarb oxamide monomethanesulfonte (Nelfinavir, marketed by Agouron as VIRACEPT®), N-(2(R)-hydroxy-1(S)-indanyl)-2(R)-phenylmethyl-4-(S)-hydroxy-5-(1-(4-(2-benzo[b]furanylmethyl)-2(S)-N′(t-butcarboxamido)-piperazinyl))-pentaneamide (See U.S. Pat. No. 5,646,148), N-(2(R)-hydroxy-1(S)-indanyl)2(R)-phenmethyl-4-(S)-hydroxy-5-(1-(4-(3-pyridymethyl)-2(S)-N′-(t-butcarboxamido)-piperazinyl))-pentaneamide (Indinavir, Marketed by Merck as CRIXIVAN®), 4-amino-N-((2 syn,3S)-2-hydroxy-4-phenyl-3-((S)-tetrahydrofuran-3-yloxycarbonylamino)-butyl)-N-isobutyl-benzensulfonamide (amprenavir, see U.S. Pat. No. 5,585,397), and N-tert-butyl-decahydro-2-[2(R)-hydroxy-4-phenyl-3(S)-[[N-(2-quinolycarbonyl)-L-asparaginyl]amino]butyl]-(4aS,8aS)-isoquinoline-3(S)-carboxamide (Saquinavir, marketed by Roche Laboratories as INVIRASE®).

Examples of suitable HIV integrase inhibitors are disclosed in U.S. Pat. Nos. 6,110,716; 6,124,327; and 6,245,806, which are incorporated herein by reference. Various other antiviral agents can also be used in a combination therapy with a R-NSAID according to the present invention, including, but not limited to, 9-(2-hydroxyrthoxymethyl)guanine (acyclovir), 2-amino-9-(2-hydroxyethoxymethyl)purine, suramin, ribavirin, antimoniotungstate (HPA-23), interferon, interleukin II, and phosphonoformate (Foscamet). In addition, other medications such as levamisol or thymosin which would stimulate lymphocyte growth and/or function may also be employed.

Examples of HIV fusion inhibitors include antibodies against HIV envelope proteins (e.g., gp120, gp41) and peptides derived from the HIV envelope proteins. For example, a gp41-derived peptide called T-20 (Trimeris Inc., Durham, N.C.) has been shown to be effective in treating HIV infection in a phase III clinical trial.

Any suitable pharmaceutically acceptable derivatives of the above compounds may also be used including pharmaceutically acceptable salts and esters thereof.

In another aspect of the present invention, a method for treating HBV infection or preventing hepatitis B is provided by administering to a patient in need of treatment a composition containing a therapeutically effective amount of the R-enantiomer of an NSAID and substantially free of the corresponding S-enantiomer. Preferably, the R-enantiomer is R-flurbiprofen.

As used herein, the term “HBV infection” generally encompasses infection of a human by any strain or serotype of hepatitis B virus, including acute hepatitis B infection and chronic hepatitis B infection. Thus, treating HBV infection means the treatment of a person who is a carrier of any strain or serotype of hepatitis B virus or a person who is diagnosed of active hepatitis B to reduce the UBV viral load in the person or to alleviate one or more symptoms associated with HBV infection and/or hepatitis B, including, e.g., nausea and vomiting, loss of appetite, fatigue, muscle and joint aches, elevated transaminase blood levels, increased prothrombin time, jaundice (yellow discoloration of the eyes and body) and dark urine. A carrier of HBV may be identified by any methods known in the art. For example, a person can be identified as HBV carrier on the basis that the person is anti-HBV antibody positive (e.g., based on hepatitis B core antibody or hepatitis B surface antibody), or is HBV-positive (e.g., based on hepatitis B surface antigens (HBeAg or HbsAg) or HBV RNA or DNA) or has symptoms of hepatitis B infection or hepatitis B. That is, “treating HBV infection” should be understood as treating a patient who is at any one of the several stages of HBV infection progression. In addition, the term “treating HBV infection” will also encompass treating suspected infection by HBV after suspected past exposure to HBV by, e.g., contact with HIBV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HBV infection” will also encompass treating a person who is free of HBV infection but is believed to be at risk of infection by HBV.

The term “preventing hepatitis B” as used herein means preventing in a patient who has HBV infection or is suspected to have HBV infection or is at risk of HBV infection from developing hepatitis B (which are characterized by more serious hepatitis-defining symptoms), cirrhosis, or hepatocellular carcinoma.

In yet another aspect of the present invention, a method for treating HCV infection or preventing hepatitis C is provided by administering to a patient in need of treatment a composition containing a therapeutically effective amount of the R-enantiomer of an NSAID and substantially free of the corresponding S-enantiomer. Preferably, the R-enantiomer is R-flurbiprofen.

As used herein, the term “HCV infection” generally encompasses infection of a human by any types or subtypes of hepatitis C virus, including acute hepatitis C infection and chronic hepatitis C infection. Thus, treating HCV infection means the treatment of a person who is a carrier of any types or subtypes of hepatitis C virus or a person who is diagnosed of active hepatitis C to reduce the HCV viral load in the person or to alleviate one or more symptoms associated with HCV infection and/or hepatitis C. A carrier of HCV may be identified by any methods known in the art. For example, a person can be identified as HCV carrier on the basis that the person is anti-HCV antibody positive, or is HCV-positive (e.g., based on HCV RNA or DNA) or has symptoms of hepatitis C infection or hepatitis C (e.g., elevated serum transaminases). That is, “treating HCV infection” should be understood as treating a patient who is at any one of the several stages of HCV infection progression. In addition, the term “treating HCV infection” will also encompass treating suspected infection by HCV after suspected past exposure to HCV by, e.g., contact with HCV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HCV infection” will also encompass treating a person who is free of HCV infection but is believed to be at risk of infection by HCV. The term of “preventing HCV” as used herein means preventing in a patient who has HCV infection or is suspected to have HCV infection or is at risk of HCV infection from developing hepatitis C (which is characterized by more serious hepatitis-defining symptoms), cirrhosis, or hepatocellular carcinoma.

The active compounds of this invention are typically administered in a pharmaceutically acceptable carrier through any appropriate routes such as parenteral, oral, or topical administration. The active compounds of this invention are administered at a therapeutically effective amount to achieve the desired therapeutic effect without causing any serious adverse effects in the patient treated.

Generally, the toxicity profile and therapeutic efficacy of the therapeutic agents can be determined by standard pharmaceutical procedures in suitable cell models or animal models. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

Typically, a R-NSAID such as R-flurbiprofen can be effective at an amount of from about 0.05 mg to about 4000 mg per day, or 10 mg to about 4000 mg per day, preferably from about 50 mg to about 2000 mg per day. However, the amount can vary with the body weight of the patient treated and the state of disease conditions. The active ingredient may be administered at once, or may be divided into a number of smaller doses to be administered at predetermined intervals of time. The suitable dosage unit for each administration of R-NSAID such as R-flurbiprofen can be, e.g., from about 0.1 mg to about 2000 mg, preferably from about 50 mg to about 1000 mg, more preferably from about 100 mg to about 800 mg.

In the case of combination therapy, a therapeutically effective amount of another anti-HIV compound can be administered in a separate pharmaceutical composition, or alternatively included in the pharmaceutical composition according to the present invention which contains an optically pure R-NSAID. The pharmacology and toxicology of many of such other anti-HIV compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds according to this invention can be administered to patients to be treated through any suitable routes of administration. Advantageously, the active compounds are delivered to the patient parenterally, i.e., by intravenous, intramuscular, intraperiotoneal, intracisternal, subcutaneous, or intraarticular injection or infusion.

For parenteral administration, the active compounds can be formulated into solutions or suspensions, or in lyophilized forms for conversion into solutions or suspensions before use. Sterile water, physiological saline, e.g., phosphate buffered saline (PBS) can be used conveniently as the pharmaceutically acceptable carriers or diluents. Conventional solvents, surfactants, stabilizers, pH balancing buffers, anti-bacteria agents, and antioxidants can all be used in the parenteral formulations, including but not limited to acetates, citrates or phosphates buffers, sodium chloride, dextrose, fixed oils, glycerine, polyethylene glycol, propylene glycol, benzyl alcohol, methyl parabens, ascorbic acid, sodium bisulfite, and the like. The parenteral formulation can be stored in any conventional containers such as vials, ampoules, and syringes.

The active compounds can also be delivered orally in enclosed gelatin capsules or compressed tablets. Capsules and tablets can be prepared in any conventional techniques. For example, the active compounds can be incorporated into a formulation which includes pharmaceutically acceptable carriers such as excipients (e.g., starch, lactose), binders (e.g., gelatin, cellulose, gum tragacanth), disintegrating agents (e.g., alginate, Primogel, and corn starch), lubricants (e.g., magnesium stearate, silicon dioxide), and sweetening or flavoring agents (e.g., glucose, sucrose, saccharin, methyl salicylate, and peppermint). Various coatings can also be prepared for the capsules and tablets to modify the flavors, tastes, colors, and shapes of the capsules and tablets. In addition, liquid carriers such as fatty oil can also be included in capsules.

Other forms of oral formulations such as chewing gum, suspension, syrup, wafer, elixir, and the like can also be prepared containing the active compounds used in this invention. Various modifying agents for flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered topically through rectal, vaginal, nasal, bucal, or mucosal applications. Topical formulations are generally known in the art including creams, gels, ointments, lotions, powders, pastes, suspensions, sprays, drops and aerosols. Typically, topical formulations include one or more thickening agents, humectants, and/or emollients including but not limited to xanthan gum, petrolatum, beeswax, or polyethylene glycol, sorbitol, mineral oil, lanolin, squalene, and the like.

A special form of topical administration is delivery by a transdermal patch. Methods for preparing transdermal patches are disclosed, e.g., in Brown, et al., Annual Review of Medicine, 39:221-229 (1988), which is incorporated herein by reference.

The active compounds can also be delivered by subcutaneous implantation for sustained release. This may be accomplished by using aseptic techniques to surgically implant the active compounds in any suitable formulation into the subcutaneous space of the anterior abdominal wall. See, e.g., Wilson et al., J. Clin. Psych. 45:242-247 (1984). Sustained release can be achieved by incorporating the active ingredients into a special carrier such as a hydrogel. Typically, a hydrogel is a network of high molecular weight biocompatible polymers, which can swell in water to form a gel like material. Hydrogels are generally known in the art. For example, hydrogels made of polyethylene glycols, or collagen, or poly(glycolic-co-L-lactic acid) are suitable for this invention. See, e.g., Phillips et al., J. Pharmaceut. Sci. 73:1718-1720 (1984).

The active compounds can also be conjugated, i.e., covalently linked, to a water soluble non-immunogenic high molecular weight polymer to form a polymer conjugate. Advantageously, such polymers, e.g., polyethylene glycol, can impart solubility, stability, and reduced immunogenicity to the active compounds. As a result, the active compound in the conjugate when administered to a patient, can have a longer half-life in the body, and exhibit better efficacy. PEGylated proteins are currently being used in protein replacement therapies and for other therapeutic uses. For example, PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). A general review of PEG-protein conjugates with clinical efficacy can be found in, e.g., Burnham, Am. J. Hosp. Pharm., 15:210-218 (1994). Preferably, the covalent linkage between the polymer and the active compound is hydrolytically degradable and is susceptible to hydrolysis under physiological conditions. Such conjugates are known as “prodrugs” and the polymer in the conjugate can be readily cleaved off inside the body, releasing the free active compounds.

Alternatively, other forms controlled release or protection including microcapsules and nanocapsules generally known in the art, and hydrogels described above can all be utilized in oral, parenteral, topical, and subcutaneous administration of the active compounds.

Another preferable delivery form is using liposomes as carrier. Liposomes are micelles formed from various lipids such as cholesterol, phospholipids, fatty acids, and derivatives thereof. Active compounds can be enclosed within such micelles. Methods for preparing liposomal suspensions containing active ingredients therein are generally known in the art and are disclosed in, e.g., U.S. Pat. No. 4,522,811, and Prescott, Ed., Methods in Cell Biology, Volume XIV, Academic Press, New York, N.Y. (1976), p. 33 et seq., both of which are incorporated herein by reference. Several anticancer drugs delivered in the form of liposomes are known in the art and are commercially available from Liposome Inc. of Princeton, N.J., U.S.A. It has been shown that liposomes can reduce the toxicity of the active compounds, and increase their stability.

### EXAMPLES

The following examples demonstrate the anti-HIV effect of R-flurbiprofen. R-flurbiprofen was supplied in powder form from Catalytica Pharmaceutical Inc., Greenville, N.C. and was solubilized in DMSO to yield a 200 mM stock solution. For these studies, the compound was tested at a high-test concentration of 1000 μM along with eight serial half-logarithmic dilutions (1000 μM down to 0.01 μM). AZT was used as a positive control antiviral compound.

1. Efficacy Evaluation in Human Peripheral Blood Mononuclear Cells (PBMCs)

Fresh human blood was obtained commercially from Interstate Blood Bank, Inc. (Memphis, Tenn.). The lymphotropic clinical isolate MV-1 _{ROJO} was obtained from a pediatric patient attending the AIDS Clinic at the University of Alabama at Birmingham. The laboratory-adapted HIV-1_{IIIB} strain was propagated and tittered in fresh human PBMCs; pre-titered aliquots of HIV-1_{ROJO} and Hiv-1_{IIIB} were removed from the freezer (−80° C.) and thawed rapidly to room temperature in a biological safety cabinet immediately before use. Phytohemagglutinin (PHA-P) was obtained from Sigma (St. Louis, Mo.) and recombinant IL-2 was obtained from Amgen (San Francisco, Calif.).

2. Anti-HIV Efficacy Evaluation in Fresh Human PBMCs

Fresh human PBMCs were isolated from screened donors, seronegative for HIV and HBV. Leukophoresed blood was diluted 1:1 with Dulbecco's phosphate buffered saline (PBS), layered over 14 mL of Ficoll-Hypaque density gradient in a 50 mL centrifuge tube and then centrifuged for 30 minutes at 600×g. Banded PBMCs were aspirated from the resulting interface and subsequently washed 2× with PBS by low speed centrifugation. After the final wash, cells were enumerated by trypan blue exclusion and re-suspended at 1×10⁷ cells/mL in RPMI 1640 supplemented with 15% Fetal Bovine Serum (FBS), 2 mM L-glutamine, 4 μg/mL PHA-P. The cells were allowed to incubate for 48-72 hours at 37° C. After incubation, PBMCs were centrifuged and reset in RPMI 1640 with 15% FBS, 2 mM L-glutamine, 100 U/ml penicillin, 100 μg/mL streptomycin, 10 μg/mL gentamycin, and 20 U/mL recombinant human IL-2. PBMCs were maintained in this medium at a concentration of 1-2×10⁶ cells/mL with biweekly medium changes until used in the assay protocol.

For the standard PBMC assay, PHA-P stimulated cells from at least two normal donors were pooled, diluted in fresh medium to a final concentration of 1×10⁶ cells/mL, and plated in the interior wells of 96 well round bottom microplate at 50 μL/well (5×10⁴ cells/well). Test drug dilutions were prepared at a 2× concentration in microtiter tubes and 100 μL of each concentration was placed in appropriate wells in a standard format. 50 μL of a predetermined dilution of virus stock was placed in each test well (final MOI ≈0.1). Wells with cells and virus alone were used for virus control. Separate plates were prepared identically without virus for drug cytotoxicity studies using an XTT assay system. The PBMC cultures were maintained for seven days following infection, at which time cell-free supernate samples were collected and assayed for reverse transcriptase activity as described below.

3. Reverse Transcriptase Activity Assay

A microtiter based reverse transcriptase (RT) reaction was utilized. See Buckheit et al., AIDS Research and Human Retroviruses 7:295-302 (1991). Tritiated thymidine triphosphate (NEN) (TTP) was resuspended in distilled H₂O at 5 Ci/ml. Poly rA and oligo dT were prepared as a stock solution which was kept at −20° C. The RT reaction buffer was prepared fresh on a daily basis and consists of 125 μI 1M EGTA, 125 μl dH₂O, 110 μl 10% SDS, 50 μl 1M Tris (pH 7.4), 50 μl 1M DTT, and 40 μl 1M MgCL₂. These three solutions were mixed together in a ratio of 2 parts TTP, 1 part poly rA:oligo dT, and 1 part reaction buffer. Ten microliters of this reactions mixture was placed at a round bottom microtiter plate and 15 μl of virus containing supernatant was added and mixed. The plate was incubated at 37° C. in a water bath with a solid support to prevent submersion of the plate and incubated for 60 minutes. Following reaction, the reaction volume was spotted onto pieces of DE81 paper, washed 5 times 5 minutes each in a 5% sodium phosphate buffer, 2 times 1 minute each in distilled water, 2 times for 1 minute each in 70% ethanol, and then dried. Opti-Fluor-O (Packard) was added to each sample and incorporated radioactivity was quantified utilizing a Wallac 1450 MicroBeta Plus liquid scintillation counter.

4. Cytotoxicity Measurement by MTS Staining

At assay termination the assay plates were stained with the soluble tetrazolium-based dye MTS (CellTiter Reagent, Promega) to determine cell viability and quantify compound toxicity. MTS is metabolized by the mitochondria enzymes of metabolically active cells to yield a soluble formazan product, allowing the rapid quantitative analysis cell viability and compound cytotoxicity. The MTS is a stable solution that does not require preparation before use. At termination of the assay, 20 μl of MTS reagent was added per well. The wells were incubated overnight for the HIV cytoprotection assay at 37° C. The incubation intervals were chosen based on empirically determined times for optimal dye reduction in each cell type. Adhesive plate sealers were used in place of the lids, the sealed plate was inverted several times to mix the soluble formazan product and the plate was read spectrophotometrically at 490 nm with a Molecular Devices Vmax plate reader.

5. Data Analysis

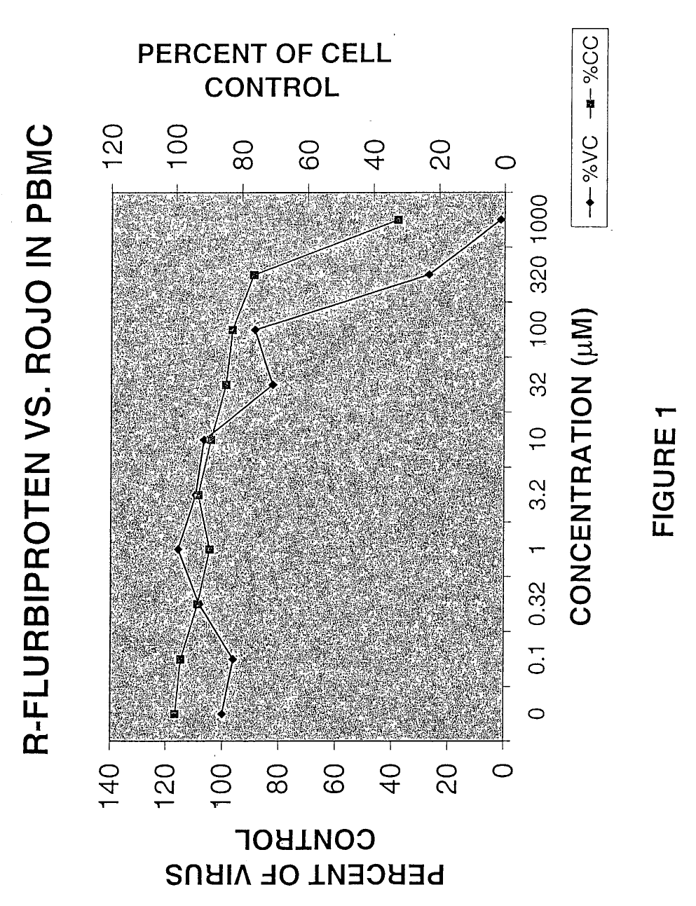

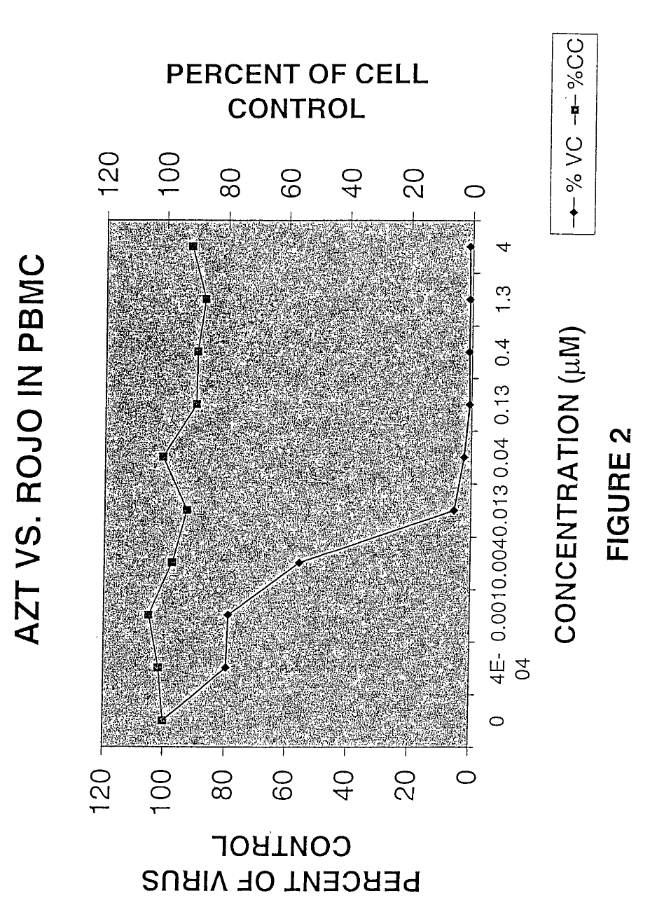

Indices including % CPE Reduction, % Cell Viability, IC₅₀, TC₅₀, and others were calculated and summarized in Table 1 below. The graphical results summary is displayed in FIGS. 1 and 2. AZT was evaluated in parallel as a relevant positive control compound in the anti-HIV assay.

TABLE 1

| Compound Name | IC₅₀ (μM) | TC₅₀ (μM) | Therapeutic Index |
|---|---|---|---|
| R-flurbiprofen | 238.1 | 730.9 | 3.1 |
| AZT | 0.01 | >4 | >400.00 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method for treating HIV infection in a patient, comprising:
administering to the patient a composition comprising a therapeutically effective amount of a R-NSAID, wherein said composition is substantially free of the S-enantiomer of said R-NSAID.

2. The method of claim 1, wherein said R-NSAID is an arylpropionic acid or a pharmaceutically acceptable salt or ester thereof.

3. The method of claim 2, wherein said R-NSAlD is selected from the group consisting of R-flurbiprofen, R-ketoprofen, R-naproxen, R-tiaprofenic acid, R-suprofen, R-carprofen, R-pirprofen, R-indoprofen, and R-benoxaprofen.

4. The method of claim 1, wherein said R-NSAID is R-ketorolac.

5. The method of claim 1, wherein said R-NSAID is R-etodolac.

6. A method for treating HIV infection in a patient, comprising:
administering to the patient a composition comprising a therapeutically effective amount of R-flurbiprofen or a pharmaceutically acceptable salt or ester thereof, wherein said composition is substantially free of S-flurbiprofen or a pharmaceutically acceptable salt or ester thereof.

7. The method of claim 6, wherein the ratio of the R-flurbiprofen or a pharmaceutically acceptable salt or ester thereof to the S-flurbiprofen or a pharmaceutically acceptable salt or ester thereof is at least 90:10 by weight.

8. The method of claim 6, wherein the ratio of the R-flurbiprofen or a pharmaceutically acceptable salt or ester thereof to the S-flurbiprofen or a pharmaceutically acceptable salt or ester thereof is at least 99:1 by weight.

9. The method of claim 6, wherein the composition contains from about 0.1 mg to 2000 mg of R-flurbiprofen or a pharmaceutically acceptable salt or ester thereof.

10. The method of claim 6, wherein from about 10 mg to 4000 mg per day of R-flurbiprofen or a pharmaceutically acceptable salt or ester thereof is administered to the patient.

11. The method of claim 6, wherein said composition further comprises one or more other anti-HIV compounds.

12. The method of claim 11, wherein said one or more anti-HIV compounds are selected from the group consisting of Zidovudine, Lamivudine, Stavudine, DMP-266, Ritonavir, Nelfinavir, Abacavir, Indinavir, 141-W94, Delavirdine, Indinavir, and Saquinavir, Tenofovir and T-20.

13. The method of claim 6, wherein said pharmaceutical acceptable salt is an alkali metal salt, alkaline earth salt, or ammonium salt.

14. The method of claim 6, wherein said ester is an alkyl ester of from 1 to 6 carbon atoms.

15. A composition useful for the treatment of HIV infection comprising:
a therapeutically effective amount of R-flurbiprofen or a pharmaceutically acceptable salt or ester thereof; and
an anti-HIV compound selected from the group consisting of HIV reverse transcriptase inhibitors, HIV protease inhibitors, HIV integrase inhibitors and HIV fusion inhibitors, wherein said composition is substantially free of S-flurbiprofen or a pharmaceutically acceptable salt or ester thereof.

16. The composition according to claim 15, wherein said anti-HIV compound is selected from the group consisting of Zidovudine, Lamivudine, Stavudine, DMP-266, Ritonavir, Nelfinavir, Abacavir, Indinavir, 141-W94, Delavirdine, Indinavir, and Saquinavir, Tenofovir and T-20.

17. A method for preventing AIDS, comprising:
administering to an individual in need of such prevention a composition comprising a prophylactically effective amount of a R-NSAID, wherein said composition is substantially free of the S-enantiomer of said R-NSAID.

18. The method of claim 17, wherein said R-NSAID is an arylpropionic acid or a pharmaceutically acceptable salt or ester thereof.

19. The method of claim 18, wherein said R-NSAID is R-flurobiprofen or a pharmaceutically acceptable salt or ester thereof.

20. The method of claim 19, wherein the composition further comprises an anti-HIV compound selected from the group consisting of HIV reverse transcriptase inhibitors, HIV protease inhibitors, HIV integrase inhibitors and HIV fusion inhibitors.

21. A method for identifying an anti-HIV compound, comprising:
providing a R-NSAID that is an arylpropionic acid selected from the group consisting of R-flurbiprofen, R-ketoprofen, R-naproxen, R-tiaprofenic acid, R-suprofen, R-carprofen, R-pirprofen, R-indoprofen, and R-benoxaprofen;
modifying the R-NSAlD to provide a R-NSAID derivative by (1) altering the position of the propionic acid group, (2) altering the position or type of substituents (other than the propionic acid group) on either of the phenyl rings, or (3) altering the bond connecting the two phenyl rings, or performing any combination of (1), (2) and (3); and
determining the effect of said R-NSAID derivative on HIV propagation.

22. The method of claim 21, wherein said R-NSAID is R-flurbiprofen.
